(19) United States
(12) Patent Application Publication
(10) Pub. No.: US 2013/0338648 A1
(43) Pub. Date: Dec. 19, 2013

(54) OPHTHALMIC LASER SURGICAL APPARATUS

(71) Applicant: NIDEK CO., LTD., Gamagori-shi (JP)

(72) Inventors: Masaaki HANEBUCHI, Gamagori-shi (JP); Masaki TANAKA, Gamagori-shi (JP)

(73) Assignee: NIDEK CO., LTD., Gamagori-shi (JP)

(21) Appl. No.: 13/907,172

(22) Filed: May 31, 2013

(30) Foreign Application Priority Data
Jun. 2, 2012 (JP) 2012-126627

(51) Int. Cl. A61F 9/008

(52) U.S. Cl. CPC A61F 9/008
USPC 606/4

(57) ABSTRACT

An ophthalmic laser surgical apparatus includes a laser irradiation optical system configured to form a spot of laser light on a patient's eye and move the position of the spot three-dimensionally, a unit configured to obtain a tomographic image of the patient's eye, a unit configured to obtain control information of the laser irradiation optical system, the control information corresponding to a discriminative portion in the tomographic image, a unit configured to determine a surgical site based on the tomographic image, a unit configured to, based on the control information, generate control information of the laser irradiation optical system for irradiating the surgical site with the laser, and a unit configured to control the laser irradiation optical system based on the control information to allow the laser irradiation optical system to apply a predetermined surgical site of the patient's eye with the laser light.

### CROSS-REFERENCE TO RELATED APPLICATION

This application is based on Japanese Patent Application No. 2012-126627 filed with the Japan Patent Office on Jun. 2, 2012, the entire content of which is hereby incorporated by reference.

### BACKGROUND

1. Technical Field

The present disclosure relates to an ophthalmic laser surgical apparatus for irradiating a patient's eye with laser light, cutting a tissue, and the like.

2. Related Art

In recent years, a technique for cutting (fragmenting) a tissue such as the crystalline lens of a patient's eye (an eye to be operated) using femtosecond order pulse laser light (an ultrashort pulse laser beam) has been proposed (see, for example, JP-T-2010-538700 (Patent Document 1)). In the apparatus of Patent Document 1, minute plasma is generated at a target position (laser spot position) of the crystalline lens to treat a cataract. Consequently, the crystalline lens tissue is mechanically cut and fragmented. After the fragmented tissue is removed, an intraocular lens or the like is inserted into the eye. Consequently, the cataract is treated. In such an apparatus, a tomographic image of the patient's eye is used to determine a laser irradiation position. For example, a tomographic image of the vicinity of the crystalline lens of the patient's eye is obtained using an optical coherence tomography (OCT) unit. The position of the front surface (anterior capsule) of the crystalline lens in the tomographic image, and the like are referred to and consequently a laser irradiation position (surgical site) is determined.

### SUMMARY

An ophthalmic laser surgical apparatus includes: a laser light source configured to emit surgical laser light; a laser irradiation optical system including a condensing optical system configured to form a spot of the laser light on a patient's eye by condensing the laser light, and a moving optical system configured to move a position of the spot three-dimensionally; a tomographic image acquisition unit configured to obtain a tomographic image of the patient's eye; a reference irradiation control information acquisition unit configured to obtain, as reference irradiation control information, control information of the laser irradiation optical system, the control information corresponding to at least one discriminative portion included in the tomographic image; a surgical site determining unit configured to determine a surgical site based on the tomographic image obtained by the tomographic image acquisition unit; an irradiation control information generating unit configured to generate control information of the laser irradiation optical system for applying the laser to the surgical site determined by the surgical site determining unit based on the reference irradiation control information obtained by the reference irradiation control information acquisition unit; and an irradiation control unit configured to control the laser irradiation optical system based on the control information generated by the irradiation control information generating unit to allow the laser irradiation optical system to apply the laser light to a predetermined surgical site of the patient's eye.

### BRIEF DESCRIPTION OF DRAWINGS

FIG. 1 is a block diagram of an ophthalmic laser surgical apparatus according to an embodiment;

FIG. 2 is block diagram of a beam expander unit;

FIG. 3 is a diagram illustrating a change in the spot size of a laser spot;

FIG. 4 is a diagram illustrating a change in the position of the laser spot in a Z direction; and

FIG. 5 is a diagram illustrating a laser irradiation area of the crystalline lens.

### DETAILED DESCRIPTION

In the following detailed description, for purpose of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

In the apparatus described in Patent Document 1, information on a depth direction of a patient's eye can be easily grasped from a tomographic image obtained by an optical coherence tomography or the like. Hence, it is easy to determine a surgical site (laser irradiation position). In the tomographic image, a discriminative portion (for example, a distance from the anterior capsule to the posterior capsule) on the image can be obtained. However, in the apparatus, it is difficult to obtain the absolute positional relationship of the discriminative portion with respect to the apparatus.

An object of the present disclosure is to provide an ophthalmic laser surgical apparatus that can improve the accuracy of surgery by determining a surgical site using a tomographic image.

An ophthalmic laser surgical apparatus includes: a laser light source configured to emit surgical laser light; a laser irradiation optical system including a condensing optical system configured to form a spot of the laser light on a patient's eye by condensing the laser light, and a moving optical system configured to move a position of the spot three-dimensionally; a tomographic image acquisition unit configured to obtain a tomographic image of the patient's eye; a reference irradiation control information acquisition unit configured to obtain, as reference irradiation control information, control information of the laser irradiation optical system, the control information corresponding to at least one discriminative portion included in the tomographic image; a surgical site determining unit configured to determine a surgical site based on the tomographic image obtained by the tomographic image acquisition unit; an irradiation control information generating unit configured to generate control information of the laser irradiation optical system for applying the laser to the surgical site determined by the surgical site determining unit based on the reference irradiation control information obtained by the reference irradiation control information acquisition unit; and an irradiation control unit configured to control the laser irradiation optical system based on the control information generated by the irradiation control information generating unit to allow the laser irradiation optical system to apply the laser light to a predetermined surgical site of the patient's eye.

The ophthalmic laser surgical apparatus can define a surgical site using a tomographic image and perform surgery thereon with high accuracy.

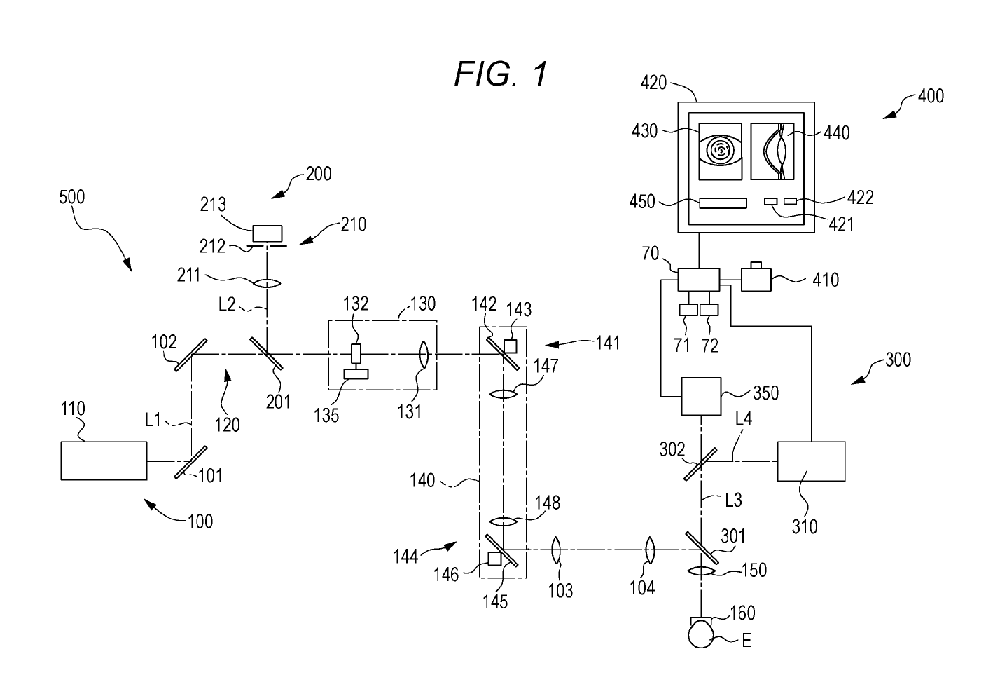

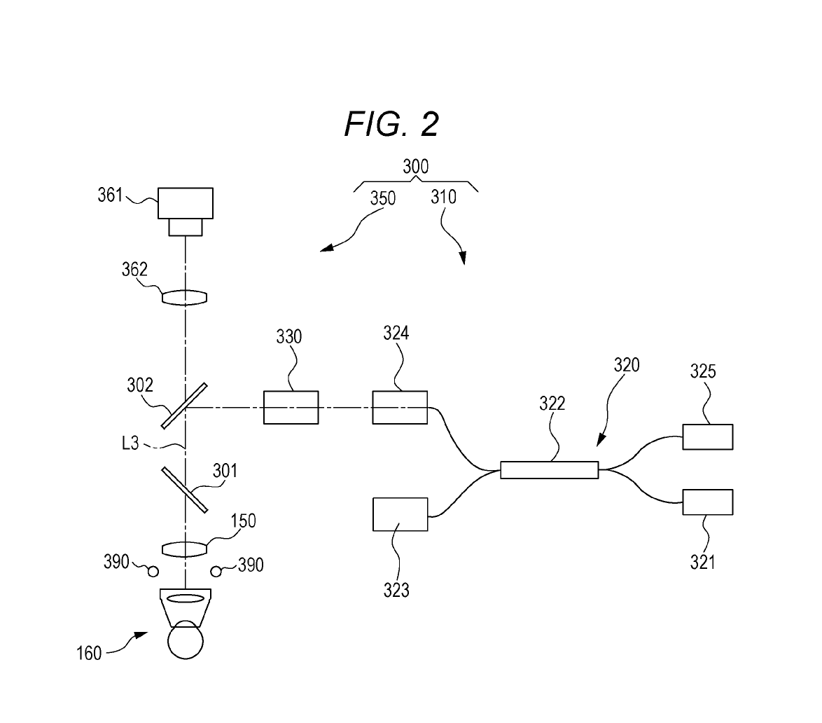

Hereinafter, an ophthalmic laser surgical apparatus (the present apparatus) according to an embodiment will be described with reference to the drawings. FIG. 1 is a schematic diagram of the present apparatus. FIG. 2 is a diagram illustrating an optical system of a tomography unit in the present apparatus. In the embodiment, the axial direction of a patient's eye E is defined as a Z direction, the horizontal direction as an X direction, and the vertical direction as a Y direction.

<Entire Configuration of the Present Apparatus>

The configuration of the present apparatus will be schematically described. In the present apparatus, an eyeball tissue (a crystalline lens LE) of the patient's eye (the eye to be operated) E is irradiated with surgical laser light (a laser beam). With the laser light, the crystalline lens is cut and fragmented.

The present apparatus (ophthalmic laser treatment apparatus) 500 mainly includes a laser irradiation unit (main body part) 100, a position detecting unit 200, an observing/photographing unit 300, an operating unit 400, and a control unit 70.

The laser irradiation unit 100 irradiates the patient's eye E with pulse laser light. The position detecting unit 200 detects the position of the patient's eye E with respect to the laser irradiation unit 100. The observing/photographing unit 300 captures an image of the anterior segment of the patient's eye E as well as a tomographic image thereof. The operating unit 400 is an operating unit for allowing an operator to operate the present apparatus 500. The control unit 70 (an irradiation control information generating unit, an irradiation control unit, and a reference irradiation control information acquisition unit) performs centralized control of the entire present apparatus 500.

The observing/photographing unit 300 includes an optical coherence tomography unit (abbreviated to OCT (Optical Coherence Tomography) unit) 310, and a front observing unit 350. The OCT unit 310 captures (obtains) a tomographic image of the patient's eye E. The front observing unit 350 captures an anterior segment image of the patient's eye E.

<Laser Irradiation Unit>

The laser irradiation unit 100 includes a laser light source unit 110, a laser delivery 120, and an eyeball fixing unit 160 for fixing an eyeball. The laser light source unit 110 emits surgical pulse laser light (a laser beam). The laser delivery 120 includes an optical component for guiding the laser light (laser light).

The laser delivery (laser irradiation optical system) 120 includes a beam expander unit 130, a scanning unit (scanning part) 140, and an objective lens 150. The beam expander unit 130 moves a laser spot in the Z direction. The scanning unit 140 moves the laser spot in the X and Y directions. The objective lens 150 is a system for concentration of light (condensing optical system) that condenses the laser light as a laser spot at a target position. The laser delivery 120 further includes various optical components for guiding the laser light.

The laser light source unit 110 is a laser light source that emits a pulse laser. Such a laser light source is used to cause an optical breakdown (photo breakdown) (cause a breakdown by nonlinear phenomena), for example, at a focal point (the position of a laser spot).

Plasma is generated at the focal point (the position of a laser spot (a spot position)). An eyeball tissue at the spot position is mechanically destroyed by the plasma (a breakdown is caused by nonlinear effects). The laser spots are connected to cut and fragment the eyeball tissue (for example, the crystalline lens). For example, a device that emits, for example, pulse laser light with a pulse width of one femtosecond to 10 nanoseconds is used as the laser light source unit 110. The laser light source unit 110 according to the embodiment emits, for example, a pulse laser in the infrared range with a pulse width of 500 femtoseconds and a center wavelength of 1030 nm. Moreover, laser light that can be emitted by the laser light source unit 110 has energy enough to cause a breakdown at a spot size of 1 to 15 μm of the laser spot.

The pulse laser to be emitted from the laser light source unit 110 has a characteristic to cause an optical breakdown at a spot position. The wavelength range of the pulse laser may be any from the infrared range to the ultraviolet range. Moreover, the pulse width of the pulse laser may be any in the range from nanoseconds to femtoseconds. For example, the laser light source unit 110 may be a device that emits pulse laser light in the ultraviolet range with a center wavelength of 355 nm and a pulse width of 10 picoseconds.

Moreover, the laser light source unit 110 has a function of adjusting energy (output) of the pulse laser light. The laser light source unit 110 can reduce the energy of the pulse laser light to suppress a breakdown at the laser spot position even if the pulse laser light is condensed to the above-mentioned spot size. Specifically, a breakdown at the laser spot position can be suppressed by reducing the energy of a laser head in the laser light source unit 110, or inserting (installing) an attenuator on an optical path of the laser light source unit 110. Moreover, in the embodiment, the energy of the laser light is adjusted and consequently the energy of surgical laser light can be adjusted. Specifically, an area of a breakdown at a spot position is adjusted. Otherwise, the turning on/off of surgical laser light (the presence or absence of laser irradiation) is adjusted (switched). The turning on/off of the laser light is switched; accordingly, the control of a moving optical system can be made constant. The turning on/off of the laser light may be switched by the control of a shutter or the laser light source (a laser head).

The attenuator may be replaced with a rotating polarizer that rotates the polarization axis of laser light or an optical shutter such as an acousto-optic modulator. The rotating polarizer rotates the polarization axis of the laser light with respect to the laser optical axis. Consequently, the energy of laser light that transmits through the rotating polarizer is adjusted. If the optical shutter is used, the energy of laser light that transmits through the optical shutter is adjusted in accordance with the control amount of frequency or the like. An attenuator may be inserted outside the laser light source unit 110 (for example, the laser optical axis in the laser irradiation optical system 120). The laser light source unit 110 is a laser light energy adjustment unit that adjusts (reduces) the energy of the pulse laser light based on a control signal.

In the laser irradiation optical system 120, the laser light source unit 110 is determined as an upstream side, and the patient's eye E as a downstream side. Mirrors 101 and 102, a hole mirror 201, the beam expander unit 130, the scanning unit 140, a lens 103, a lens 104, and a beam combiner 301 are arranged along an optical axis L1 in this order from the laser light source unit 110 toward the downstream side.

The mirrors 101 and 102 adjust the optical axis of the laser light. The hole mirror 201 is a beam splitter for separating the optical axis L1 of the laser light and an optical axis L2 of the position detecting unit. The lens 103 forms an intermediate image of the laser light. The lens 104 forms a pupil conjugate position. The beam combiner 301 multiplexes the optical axis L1 and an optical axis L3 of the observing/photographing unit 300.

The mirrors 101 and 102 are configured such that their reflecting surfaces are orthogonal to each other. The mirrors 101 and 102 are held by inclinable holding components, respectively. The reflecting surfaces of the mirrors 101 and 102 are moved and/or inclined; accordingly, it is possible to adjust the optical axis of the pulse laser light emitted from the laser light source unit 110. The mirrors 101 and 102 are adjusted to cause the optical axis of the pulse laser light to coincide with the optical axis L1.

The beam expander unit 130 (hereinafter simply referred to as the expander) includes a plurality of optical elements. The expander 130 changes divergence of a beam of the pulse laser light passing through itself. Consequently, the expander 130 can move a laser spot along the Z direction (on the optical axis L1).

The expander 130 of the embodiment includes an optical element (concave lens) 131 having a negative refractive power, an optical element (convex lens) 132 having a positive refractive power, and a driving part 135 that moves the lens 131 along the optical axis L1. The lens 131 is moved; accordingly, the divergence (divergence angle, convergence angle, and the like) of the beam that has transmitted through the lens 132 can be changed. The condensing position of the laser spot is to be changed in the Z direction in accordance with the divergence of the pulse laser light incident on the objective lens 150.

The scanning unit (optical scanner unit) 140 includes a first optical scanner (first light deflecting component) 141 for moving the laser light in the X direction and a second optical scanner 144 (a second light deflecting component) for moving the laser light in the Y direction. For example, the first optical scanner 141 has a mirror 142. The minor 142 is a deflecting component for moving the laser light in the X direction. The second optical scanner 144 has a mirror 145. The mirror 145 is a deflecting component for moving the laser light in the Y direction. Furthermore, the scanning unit 140 includes lenses 147 and 148 for conjugating the minors 142 and 145 to the pupil. For example, the first optical scanner and the second scanner may include a galvanometer minor.

The first optical scanner 141 and the second optical scanner 144 are configured to move (deflect) the laser light in directions orthogonal to each other. The mirror 142 is held by a driving part 143 rotatably around the axis. The mirror 145 is held by a driving part 146 rotatably around the axis. The minors 142 and 145 are configured such that reflecting surfaces of the minors 142 and 145 are parallel to each other (or orthogonal to each other) when both of the driving parts 143 and 146 are at their reference positions.

The focal point (primary focal point) of the lens 147 is aligned to the center of the mirror 142 (the center of rotation of the mirror surface). The focal point (secondary focal point) of the lens 148 is aligned to the center of the mirror 145 (the center of rotation of the mirror surface). Therefore, the axis of the laser light that has passed through the lens 147 becomes substantially parallel to the optical axis L1. Moreover, the laser light forms an image (forms an intermediate image) once between the lenses 147 and 148. The spot size of the intermediate image is larger than a spot size at the target position. Hence, an optical breakdown (here, plasma) does not occur at the spot position of the intermediate image. In an optical system having such a configuration, it is possible to inhibit the spread of the laser light deflected (scanned) by the first optical scanner 141 and the second optical scanner 144 in the X and Y directions. Hence, it is possible to reduce the effective diameter of an optical component downstream of the scanning unit 140. Hence, the entire present apparatus 500 can be downsized.

The scanning unit 140 has a configuration capable of scanning the laser light in the X and Y directions. The scanning unit 140 may include, for example, a polygon mirror for scanning in the X direction and a galvanometer mirror for scanning in the Y direction. Moreover, the scanning unit 140 may be configured to use a resonant mirror adapted to the X and Y directions. Moreover, the scanning unit 140 may be configured to rotate two prisms independently of each other.

In this manner, in the present apparatus 500, the laser spot is moved three-dimensionally (in the X, Y and Z directions) by the expander 130 and the scanning unit 140 in an eyeball tissue (in a target) of the patient's eye E. The expander 130 and the scanning unit 140 are included in the moving optical system. The expander 130 is arranged upstream of the scanning unit 140. Consequently, it is possible to inhibit the laser light from passing through the expander 130 after being directed to the X and Y directions. Hence, the effective diameter and size of the optical component of the expander 130 can be reduced. Furthermore, the lens 131 can be made smaller. As a consequence, it is possible to accelerate the movement of the laser spot in the Z direction.

An intermediate image is formed downstream of the lens 103; accordingly, the effective diameters and sizes of the lens 104, the beam combiner 301, and the objective lens 150, which are arranged downstream of the lens 103, can be reduced. The lens 104 forms a pupil conjugate position in the scanning unit 140 on a reflecting surface of the beam combiner 301. The beam combiner (beam splitter) 301 has a characteristic that reflects the pulse laser light while transmitting the illuminating light of the observing/photographing unit 300. The objective lens 150 is a lens arranged fixedly. The objective lens 150 causes the laser light to form an image on a target so as to have a minute laser spot of approximately 1 to 15 μm.

The eyeball fixing unit (applicator) 160 that comes into contact with the patient's eye E includes a suction ring for sucking and fixing an eyeball thereto, and a cup for covering around the anterior segment. Negative pressure is applied to the suction ring by an external suction pump and the like. Consequently, the anterior segment is suctioned to the suction ring. At the time of surgery, the cup is filled with a liquid having a refractive index substantially equal to the refractive index of the cornea. The surface of the liquid is covered with a cover. Hence, the influence of the shaking of the liquid surface is inhibited. Consequently, the refraction of the laser light at the cornea or the like is weakened. As a consequence, the laser light is suitably guided. The eyeball fixing unit 160 may be a contact lens or contact glass having a surface that comes into contact with the cornea.

Although an illustration is omitted, the laser irradiation unit 100 has an aiming light source. The aiming light source emits aiming light (aiming light) to be used for the operator to check a laser irradiation position.

<Position Detecting Unit>

The position detecting unit 200 cooperates with the control unit 70. The position detecting unit 200 obtains, as reference irradiation control information, control information of the laser irradiation optical system 120, the control information corresponding to at least one discriminative portion (tissue) included in a tomographic image to be described below. In other words, the position detecting unit 200 serves as the reference irradiation control information acquisition unit.

The position detecting unit 200 obtains position information of the patient's eye E to generate the control information of the laser irradiation unit 100 (the laser irradiation optical system 120) with respect to the patient's eye E. The position detecting unit 200 has a function of detecting a relationship between the discriminative portion of the patient's eye E and the position of the laser irradiation optical system 120. The position detecting unit 200 includes an optical system for position detection. The optical system is a confocal optical system in the embodiment. In other words, the position of a receiving part of a light receiving device 213, which is described below, and a spot position of laser light from a light source is in a substantially conjugate relationship.

At least one component of a position detecting optical system 210 also serves as at least one component of the laser irradiation optical system 120. The optical axis L2 of the position detecting optical system 210 is multiplexed by the hole mirror 201 with the optical axis L1 of the laser irradiation optical system 120. The position detecting unit 200 further includes the hole minor 201, a condensing lens 211, an aperture plate 212, and the light receiving device 213.

The hole mirror 201 is a total reflection mirror having an aperture at the center. The hole mirror 201 transmits the laser light emitted by the laser light source unit 110. The hole mirror 201 reflects the laser light reflected from the patient's eye E in a direction along the optical axis L2. The lens 211 is responsible for condensing the laser light reflected by the hole mirror 201 to the aperture of the aperture plate 212. The aperture plate 212 is a confocal aperture plate having an aperture at the center. In other words, the aperture of the aperture plate 212 is arranged at a position to be conjugated (in a confocal relationship) by the lens 211 and the like to the position of the laser spot in the patient's eye E. The light receiving device 213 is a light receiving device such as a photodiode or a photo-multiplier. The light receiving device 213 receives the laser light that has passed through the aperture of the aperture plate 212. The light receiving device 213 generates a light receiving signal in accordance with the intensity of the received laser light and transmits the signal to the control unit 70.

In the position detecting optical system 210, the laser light emitted from the laser light source unit 110 is guided by the laser irradiation optical system 120 and is condensed (forms an image) to the target position of the patient's eye E. The laser light reflected by the patient's eye E, following back through the laser irradiation optical system 120, is reflected by the hole mirror 201, and received by the light receiving device 213. The laser light reflected by the patient's eye E includes the laser scattered by the patient's eye.

At the time of position detection, the laser light emitted from the laser light source unit 110 is adjusted so as to have energy that does not cause a breakdown at the laser spot. The light receiving signal obtained by the light receiving device 213 is used as information to detect the position of the patient's eye E. In the position detecting optical system 210, the position of the laser spot is moved three-dimensionally (moved in the X, Y, and Z directions) by the expander 130 and the scanning unit 140, which share part of the optical system. Specifically, the position of the laser spot is identified from information of the moving optical system including the position of the lens 131 (information of the driving part 135), and directions of the minor 142 and 145 (information of the driving parts 143 and 146). The obtained position information is used as control information of the moving optical system.

In this manner, part of the components of the position detecting optical system 210 also serve as part of the components of the laser irradiation optical system 120. Furthermore, the laser light source unit 110 also serves as a light source for position detection. Consequently, information on a laser spot position of surgical laser light can be detected. Specifically, the laser spot is moved by the expander 130 and the scanning unit 140. At the same time, a change in the intensity of the light receiving signal of the light receiving device 213 is monitored. Consequently, information of a boundary portion (for example, the front surface of the cornea) of a tissue of the patient's eye E is detected with high accuracy. Moreover, the position detecting unit 200 can detect the position of a laser spot with high accuracy by using the confocal relationship. The hole mirror 201 may be a polarizing beam splitter. The hole minor 201 may be configured to separate irradiation light and reflected light using the action where the polarizing direction of the laser light is rotated 90 degrees on a reflecting surface.

<Observing/Photographing Unit>

The observing/photographing unit 300 includes the OCT unit 310 that obtains a tomographic image of the patient's eye E, and the front observing unit (front image acquisition unit) 350 that obtains a front image of the patient's eye E. The optical axis L3 of the observing/photographing unit 300 is made substantially coaxial (or substantially parallel) with the laser optical axis L1 by the beam combiner 301. The optical axis L3 is divided by a beam combiner 302 into an optical axis L4 of the OCT unit 310. The beam combiner 302 is a dichroic mirror. The beam combiner 302 has a characteristic that reflects measurement light of the OCT unit 310 while transmitting (the reflected light of) illuminating light for the front observing unit 350.

<Optical Tomography Unit>

The optical coherence tomography unit (OCT unit) 310 and the laser irradiation optical system 120 share the objective lens 150. The OCT unit 310 includes a coherent optical system (OCT optical system) 320 for photographing a tomographic image of (the anterior segment of) the patient's eye E (See FIG. 2).

The OCT optical system 320 irradiates the patient's eye E with measurement light. The OCT optical system 320 detects a coherent state of the measurement light reflected from the patient's eye E and reference light, by a light receiving device (a detector 325). The OCT optical system 320 includes an optical scanner 330 to change the imaging position of the patient's eye E. The optical scanner 330 is an irradiation position changing unit that changes the irradiation position of the measurement light at the fundus (patient's eye) E. The optical scanner 330 is connected to the control unit 70. The control unit 70 controls the optical scanner 330 based on the set imaging position information. Consequently, the control unit 70 obtains a tomographic image based on a light receiving signal from the detector 325.

The OCT optical system 320 has an apparatus configuration of what is called an ophthalmic optical coherence tomography. In the embodiment, the OCT optical system 320 captures at least a tomographic image of the patient's eye E before the pulse laser light is applied thereto. The OCT optical system 320 divides light (infrared light) emitted from a measurement light source 321 by a coupler 322 as a light splitter into measurement light (sample light) and reference light. The OCT optical system 320 then guides the measurement light to the patient's eye E by a measuring optical system 324 while guiding the reference light to a reference light optical system 323. The OCT optical system 320 subsequently receives, by the detector (light receiving device) 325, interference light obtained by combining the measurement light reflected by the patient's eye E and the reference light. The light splitter may be a coupler, a beam splitter, or a circulator.

The detector 325 detects a coherent state of the measurement light and the reference light. In Fourier domain OCT, spectral intensity of the interference light is detected by the detector 325. A depth profile (A-scan signal) in a predetermined area is obtained by the Fourier transform on spectral intensity data. Fourier domain OCT includes, for example, Spectral-Domain OCT (SD-OCT) and Swept-Source OCT (SS-OCT). Moreover, Fourier domain OCT may be Time-Domain OCT (TD-OCT).

In SD-OCT, the light source 321 is a low coherent light source (broadband light source). In the detector 325, a spectral optical system (spectrometer) that splits the interference light into frequency components (wavelength components) is provided. The spectrometer includes, for example, a diffraction grating and a line sensor.

In SS-OCT, the light source 321 may be a wavelength scanning light source (wavelength variable light source) that temporally changes an emission wavelength at high speeds. The detector 325 may be, for example, a single light receiving device. The light source 321 includes, for example, a light source, a fiber ring resonator, and a wavelength selective filter. The wavelength selective filter may include, for example, a combination of a diffraction grating and a polygon mirror, or a Fabry-Perot etalon.

Light emitted from the light source 321 is divided by the coupler 322 into measurement light and reference light. The measurement light is emitted into the air after passing through an optical fiber. The light is condensed to the patient's eye E through the measuring optical system 324 and the optical scanner 330. The light reflected by the patient's eye E is returned to the optical fiber through a similar optical path.

The reference optical system 323 generates reference light. The reference light is combined with reflected light obtained by the reflection of the measurement light from the patient's eye E. The reference optical system 323 may be a Michelson system or Mach-Zehnder system. The reference optical system 323 may include, for example, a catoptric system (for example, a reference mirror). The reference optical system 323 reflects the light from the coupler 322 by the catoptric system. Consequently, the reference optical system 323 returns the light to the coupler 322 again and guides the light to the detector 325. As another example, the reference optical system 323 may include a transmission optical system (for example, an optical fiber). In this case, the light from the reference optical system 322 is not returned but transmitted through, and guided to the detector 325.

The reference optical system 323 includes a component for changing a difference in optical path length between the measurement light and the reference light by moving the optical component on the reference optical path. For example, a reference mirror is moved in the optical axis direction to change the difference in optical path length. The component for changing the difference in optical path length may be arranged on the measurement optical path of the measuring optical system 324.

The OCT unit 310 includes the optical scanner 330 for deflecting the measurement light flux. The optical scanner 330 includes two galvanometer mirrors whose rotation axes are orthogonal to each other. The optical scanner 330 has a function of deflecting the measurement light flux two-dimensionally based on an instruction signal from the control unit 70. The control unit 70 controls the optical scanner 330 to scan the patient's eye E with the measurement light in the X-Y direction (transverse direction). In the embodiment, the anterior segment of the patient's eye E is scanned with the measurement light. For example, the control unit 70 linearly arranges depth information (depth information) obtained at scan positions. Consequently, the control unit 70 obtains a tomographic image (what is called a B-scan).

In this manner, the light flux emitted from the light source 321 is changed in terms of its reflection (advance) direction and scans the anterior segment in an arbitrary direction. Consequently, the tomographic position of the patient's eye E is changed.

The optical scanner 330 is configured to deflect light. The optical scanner 330 may include, for example, a reflection minor (a galvanometer mirror, a polygon minor, a resonant scanner) or may include an acousto-optic modulator (AOM) that changes (deflects) the advance direction of light, and the like.

A refraction correction process is performed on a tomographic image of the anterior segment of the patient's eye E. A detailed description thereof is omitted.

<Front Observing Unit>

The front observing unit 350 has a function of acquiring a front image of the anterior segment of the patient's eye E. In the embodiment, the front observing unit 350 captures an anterior segment image of the patient's eye E illuminated with visible light or infrared light and displays the front segment image on a monitor, which is described below. The anterior segment image is displayed on the monitor at a frame rate of approximately 30 ms. The front observing unit 350 includes an observing optical system (a front image observing optical system). The observing optical system includes a camera unit (camera) 361 having a two-dimensional imaging device, and a relay lens 362 for relaying an observation image. The front observing unit (front image observing unit) 350 and the laser irradiation optical system 120 share the objective lens 150. Moreover, an illuminating light source 390 that emits visible illuminating light or infrared light is arranged in the vicinity of the front of the patient's eye E. The camera 361 captures a front image of the anterior segment of the patient's eye E illuminated with the illuminating light from the illuminating light source 390. The photographed front image is transmitted to the control unit 70.

<Operating Unit>

The operating unit 400 includes a trigger switch 410 and a monitor 420. A trigger signal to cause the laser irradiation unit 100 to emit treatment laser light is input into the trigger switch 410. The monitor (display part) 420 displays a tomographic image and/or anterior segment image of the patient's eye E, and surgical conditions. The monitor 420 has a touchscreen function. The monitor 420 also serves as an input part for carrying out the setting of the surgical condition and/or the setting of a surgical site (laser irradiation position) on a tomographic image. The input part may be a mouse being a pointing device, and/or a keyboard being an input device for inputting numerical values, characters, and the like.

The monitor 420 includes an anterior segment image display part (anterior segment display part) 430 for displaying the anterior segment of the patient's eye E, an OCT image display part 440 for displaying a tomographic image of the anterior segment of the patient's eye E, a surgical condition display part 450 for displaying the surgical conditions, a switch 421 for stating the setting work (planning) of a surgical site, and a switch 422 for determining the specified surgical site (determine the planning).

On the OCT image display part 440, the operator specifies a surgical site (a laser irradiation area) graphically. The surgical site specified on the monitor 420 is set as a laser irradiation position on an OCT image. The control unit 70 stores the set surgical site in a memory 71. The switches 421 and 422 have a function of switching the mode of the apparatus 500 to a planning mode. The monitor 420 (the OCT image display part 440) serves as a surgical site determining unit.

On the surgical condition display part 450, the operator sets an irradiation pattern of surgical laser light to fragment (incise) the crystalline lens. There is a plurality of irradiation patterns prepared in advance. The operator selects one of the irradiation patterns. When the irradiation pattern is set by the surgical condition display part 450, the monitor 420 transmits a setting signal to the control unit 70. Laser energy at the time of surgery, the spot size of a laser spot, and the like may not be changed or may be changed (set) by the operator.

<Control System>

The control unit 70 oversees and excises control over the entire present apparatus 500. The control unit 70 includes, for example, a CPU (Central Processing Unit). The control unit 70 is connected to the laser light source unit 110, the driving part 135, the first optical scanner 141 and the second optical scanner 144, the light receiving device 213, the measurement light source 321, the reference optical system 323, the measuring optical system 324, the detector 325, the optical scanner 330, the camera 361, and the operating unit 400 (the trigger switch 410 and the monitor 420).

Moreover, the control unit 70 is connected to the memory 71. The memory 71 stores therein the surgical conditions, the irradiation patterns (patterns to move a laser spot), and the like. Moreover, the control unit 70 is connected to a buzzer 72. The buzzer 72 notifies the operator of the end of work and/or alert, and the like. The eyeball fixing unit 160 and the illuminating light source 390 are driven individually.

Before the irradiation of the surgical laser light, the control unit 70 uses the position detecting unit 200 to obtain reference irradiation control information of the laser irradiation optical system 120 (the moving optical system), the reference irradiation control information corresponding to a discriminative portion included in a tomographic image. The control unit 70 generates control information for irradiating surgical laser light, based on the set surgical site and the reference irradiation control information.

The control information of the embodiment includes the energy of laser light to be emitted from the laser light source unit 110, and the positions and directions of the optical elements of the moving optical system (the expander 130 and the scanning unit 140). The control unit 70 controls the laser light source unit 110, the expander 130, and the scanning unit 140, based on the generated control information. Consequently, the set surgical site is irradiated with the surgical laser light. The control information also includes the set irradiation pattern.

<Flow of Laser Surgery>

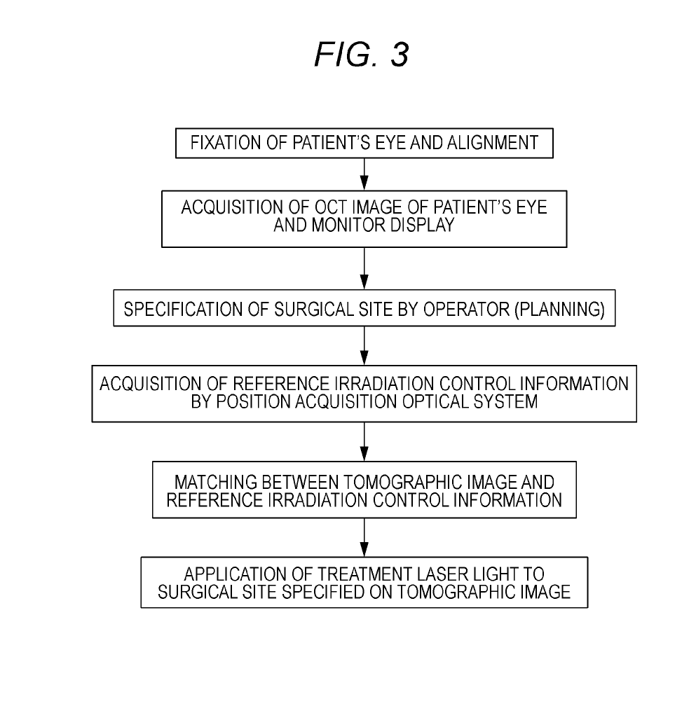

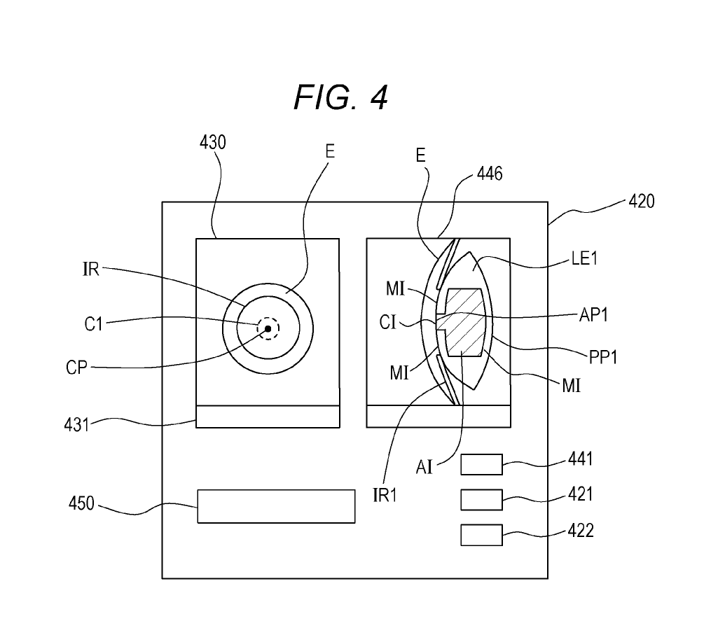

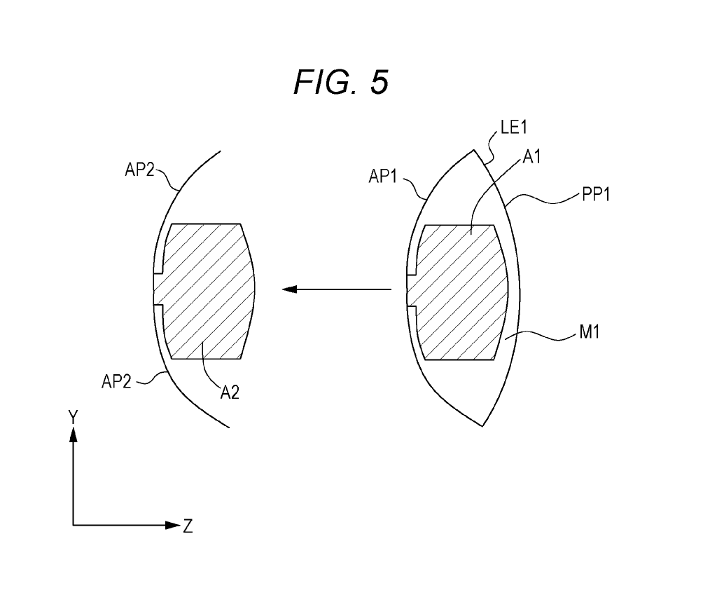

Next, the flow of laser surgery will be described. FIG. 3 is a flowchart illustrating the flow of laser surgery. Hereinafter, the operation of the present apparatus 500 and a computation process of the control unit 70 will be described with reference to the flowchart. A specific example of the association between the setting of a surgical site and laser irradiation is described below with reference to FIGS. 4 and 5.

The operator sets the surgical condition by operating the surgical condition display part 450 of the monitor 420. Here, the operator selects, for example, an irradiation pattern for fragmenting the crystalline lens. The irradiation patterns include, for example, a pattern for incising only the anterior capsule of the crystalline lens, a pattern for incising the anterior capsule and segmenting the crystalline lens nucleus (for example, into two, four, or eight), and a pattern for incising the anterior capsule and fragmenting the crystalline lens nucleus into small pieces. A signal for setting the irradiation pattern is transmitted to the control unit 70 and stored in the memory 71 (the setting of the surgical condition).

Next, the operator attaches (suctions) the suction ring of the eyeball fixing unit 160 to the patient's eye E of a patient (person to be operated) who is lying on a bed or the like. The operator places the cup of the eyeball fixing unit 160 on the cornea of the patient's eye E and fills liquid in the cup. The operator then arranges the laser irradiation unit 100, the observing/photographing unit 300, and the like above the eyeball fixing unit 160. Consequently, the central axis of the patient's eye E is aligned with the laser optical axis L1 (the fixation of the patient's eye, and alignment (the adjustment of the position of the patient's eye with respect to the apparatus).

When the alignment is complete, images of the patient's eye E are displayed in real time on the anterior segment image display part 430 and the OCT image display part 440 of the monitor 420. The control unit 70 controls the front observing unit 350 to obtain an anterior segment image. The anterior segment image obtained by the camera unit 361 is transmitted to the control unit 70. The anterior segment image is displayed on the anterior segment image display part 430 while updated at a constant frame rate. Moreover, the control unit 70 controls the OCT unit (OCT image photographing unit) 310 to obtain an OCT image. The control unit 70 displays the OCT image (the anterior segment tomographic image) on the OCT image display part 440 based on the light receiving signal obtained by the detector 325. The control unit 70 displays the OCT image while updating the OCT image at a constant frame rate (the acquisition of an anterior segment image and OCT image of the patient's eye, and monitor display).

When the operator operates (touches) the switch 421, an instruction signal to start planning is transmitted to the control unit 70. Then, the control unit 70 displays a still image of the patient's eye E on the anterior segment image display part 430 based on the instruction signal. Furthermore, the control unit 70 displays a still image of the OCT image on the OCT image display part 440. At this point, the control unit 70 obtains a plurality of OCT images corresponding to different B-scan directions and stores the OCT images in the memory 71. The control unit 70 obtains a plurality of OCT images while, for example, changing the Y direction by 30 degrees in B-scans. For example, six OCT images corresponding to the Y direction in B-scans at 90, 120, 150, 180, 210, and 240 degrees are obtained. It is advantageous to obtain a plurality of tomographic images as described above because three-dimensional data of the anterior segment can be obtained based on the tomographic images. The control unit 70 may obtain three-dimensional data of the anterior segment formed of a plurality of tomographic images adjacent to each other by raster scanning in the X and Y directions. It is preferred that the control unit 70 be configured to obtain at least two tomographic images orthogonal to each other, including the optical axis of laser irradiation. In this case, the control unit 70 and the position detecting unit 200 obtain the reference irradiation control information based on the plurality of tomographic images.

The control unit 70 enables the operator to specify a surgical site using the anterior segment image display part 430 and the OCT image display part 440. The operator specifies an area of an incision in the anterior capsule (the diameter of a circle and the like) for the anterior segment image displayed on the anterior segment display part 430. The control unit 70 displays on the anterior segment image display part 430 the pupil center and the position (circle) of the incision in the anterior capsule by image processing on the anterior segment image.

Moreover, the operator specifies an area (surgical site) of the crystalline lens to be fragmented or incised for the OCT image displayed on the OCT image display part 440. The control unit 70 specifies a fragmentation area by image processing on the OCT image (the details are described below). The specification of the surgical site (area) of the crystalline lens leads to the determination of an area in the depth direction (the Z direction). If the switch 422 is operated, the planning at the current stage is determined. The control unit 70 stores in the memory 71 the information on the position of the incision in the anterior capsule in the anterior segment image and the information on the surgical site (area) on the OCT image as surgical site information (planning information) (the setting of a surgical site).

The control unit 70 obtains a positional relationship between the laser irradiation optical system 120 and the discriminative portion of the patient's eye. For this purpose, the control unit 70 controls the position detecting unit 200. Cited as discriminative portions of the patient's eye are, for example, the front or back surface of the cornea, the iris, or the anterior or posterior surface of the crystalline lens.

The control unit 70 reduces the energy of the laser light source unit 110 in the control of the position detecting unit 200. The energy of laser light emitted from the laser irradiation unit 100 (here, the laser light source unit 110) is switched between energy for surgery that causes a breakdown at a laser spot position and energy for position detection that does not cause a breakdown at a laser spot position. Moreover, the control unit 70 controls the position detecting unit 200 (the light receiving device 223, the driving part 135, and the first optical scanner 141 and the second optical scanner 144) and receives the reflected light from the laser spot position of the patient's eye E by the light receiving device 223.

The control unit 70 obtains (detects) three-dimensional luminance information on the patient's eye E based on the light receiving signal of the light receiving device 223 and the information of the driving part 135, the first optical scanner 141, and the second optical scanner 144. The control unit 70 obtains, as the reference irradiation control information, control information of the laser light source unit 110 and the moving optical system (the driving part 135, and the optical scanners 141 and 144) for applying laser light to a position corresponding to the discriminative portion of the patient's eye based on (the intensity of) the light receiving signal and the information of the moving optical system. In other words, the control unit 70 obtains (detects) the discriminative portion (in the following example, the absolute position of the anterior lens capsule) of the patient's eye E. The control unit 70 obtains the position and shape of the curved surface of the anterior lens capsule from the detection result. The control unit 70 obtains, as the reference irradiation control information, control information of the laser light source unit 110 and the moving optical system for applying laser light to the obtained anterior capsule and stores the control information in the memory 71 (the acquisition of the reference irradiation control information).

The control unit 70 associates the reference irradiation control information with the surgical site information. The control unit 60 then generates control information of the laser irradiation unit 100 for irradiating the surgical site with surgical laser.

When the surgical site is irradiated with surgical laser, the control unit 70 controls, for example, the moving optical system to move the condensing position three-dimensionally. The control unit 70 applies laser light by the laser light source unit 110 when the condensing position is in an area corresponding to the surgical site. On the other hand, the control unit 70 stops laser light irradiation when the condensing position is in another area. In this case, for example, control information on the laser light source unit 110 and the moving optical system is predetermined as the control information of the laser irradiation unit 100.

The control unit 70 matches the reference irradiation control information corresponding to the discriminative portion (for example, the anterior capsule) with the discriminative portion (for example, the anterior capsule) in a tomographic image. Based on the matching result, the control unit 70 associates the control information of the moving optical system (for example, the positions, directions, and the like of the optical elements of the moving optical system (the expander 130 and the scanning unit 140) with the coordinate position in the tomographic image. Once they are associated, the control unit 70 obtains control information of the laser irradiation unit 100 for applying surgical laser to the area set in the tomographic image.

When the matching is complete, the control unit 70 controls the buzzer 72 to notify the operator of the completion of the preparation of surgery. When the trigger switch 410 is operated, the control unit 70 starts laser irradiation. The control unit 70 controls the laser light source unit 110, the expander 130, and the scanning unit 140 based on the control information of the laser irradiation unit 100, the irradiation pattern, and the surgical condition. The patient's eye E is irradiated with a laser; accordingly, the crystalline lens is fragmented and incised (the application of treatment laser light to a surgical site specified on a tomographic image).

In the above description, the surgical site determined by the planning is associated with the reference irradiation control information to obtain the control information, but not limited thereto. It is sufficient if the control of the control unit 70 enables the obtainment of the control information of the laser irradiation unit 100 for applying a surgical laser. The control unit 70 may be configured to previously obtain the reference irradiation control information by the position detecting unit 200 or the like before the setting of a surgical site. In this case, when the operator works on the setting of a surgical site, the control information is generated.

In the above description, the planning is carried out at the time of surgery but not limited thereto. Alternatively, the planning may be carried out before laser irradiation. The planning may be carried out by the present apparatus 500, for example, several days before surgery, and the planning data may be read at the time of surgery. Moreover, the planning may be carried out by another apparatus (a filing system to display a tomographic image, or the like). In this case, the present apparatus 500 (the control unit 70) reads the planning data. In such an example, the above-mentioned planning is the work of checking.

<Monitor Display and Planning>

Next, specification of a surgical site on the monitor and image processing will be described. FIG. 4 illustrates a display screen of the monitor 420. FIG. 5 is a diagram illustrating position correction. Processing after the switch 421 is operated is described here.

An anterior segment image of the patient's eye E is being displayed on the anterior segment image display part 430. An incision size display field 431 that represents the size of a circular incision in the anterior capsule is arranged under the anterior segment image display part 430. The control unit 70 obtains the pupil center in the anterior segment image by image processing. The control unit 70 obtains the position of the pupil center from the shape of an iris IR in the anterior segment image. The control unit 70 displays a pupil center CP in the anterior segment image when the anterior segment image display part 430 is operated. The control unit 70 then displays a circle C1 centering the pupil center CP in the anterior segment image. The circle C1 indicates the incision position of the anterior capsule by laser irradiation. The diameter of the circle C1 is displayed so as to be a diameter shown in the incision size display field 431. The operator operates the incision size display field 431 to display an increase/decrease switch (not shown). The incision size can be changed by the increase/decrease switch. The incision size displayed in the incision size display field 431 is stored in the memory 71.

The shape of an incision in the anterior capsule is not limited to a circle, but may be a predetermined figure (for example, an ellipse). Moreover, the circle C1 (the center thereof) may be decentered from the pupil center CP (shifted in the X and Y directions). In this case, the anterior segment image display part 430 (the monitor 420) may be configured, for example, to display a field to input an off-center distance of the circle C1 and to accept input of a distance desired by the operator. Moreover, the center of the circle C1 is not limited to the pupil center, but may be the apex of the crystalline lens or the like. The apex of the crystalline lens is obtained by extracting the apex of the anterior capsule in a plurality of OCT images.

A tomographic image of the patient's eye E in the Y direction, passing through the central axis of the laser irradiation optical system (the central axis of the laser light), is displayed on the OCT image display part 440. A margin display field 441 that displays a margin amount in the fragmentation (incision) of the crystalline lens nucleus is arranged below the OCT image display part 440. The margin described here is an area, to which laser light is not applied, provided in the front of a posterior capsule PP1 of the crystalline lens (in the rear of an anterior capsule AP1) so as not to damage the lens capsule with the laser light. The margin amount is set, for example, to 50 to 1000 μm (here, it is assumed to be 500 μm). If the operator operates the margin display field 441, an increase/decrease switch (not shown) is displayed. The operator can change the margin amount with the increase/decrease switch. The margin amount displayed in the margin display field 441 is stored in the memory 71.

An area A1 indicating the surgical site is displayed on the OCT image display part 440. The control unit 70 performs image processing on image data forming an OCT image. Consequently, the control unit 70 seeks the area A1 indicating the surgical site (hereinafter abbreviated to the area A1). The control unit 70 highlights an area corresponding to the area A1 on the OCT image.

The control unit 70 detects from the OCT image the position of the anterior capsule AP1 of a crystalline lens LE1, the position of the posterior capsule PP1 of the crystalline lens LE1, and the iris IR in front of the vicinity of the anterior capsule AP1. The control unit 70 defines an area between the anterior capsule AP1 and the posterior capsule PP1 based on the margin stored in the memory 71. The control unit 70 defines an area excluding a place where the iris IR covers the anterior capsule AP1 (an area in the rear of the iris IR). At this point, the control unit 70 does not define the margin at a position corresponding to the circle C1. Moreover, the control unit 70 displays a margin area M1 on the crystalline lens LE1 (or in the inside thereof). The control unit 70 sets the area M1 in the rear of the anterior capsule AP1 by the size of the margin. On the other hand, the control unit 70 does not define the margin in the area corresponding to the circle C1. Moreover, the control unit 70 defines the area M1 frontward of the posterior capsule PP1 by the size of the margin.

The area A1 can be changed (finely adjusted) by the operation of the operator. The operator can change a boundary line of the area A1 at will. The operator touches and drags the boundary line of the area A1 and consequently can change the shape of the area A1. At this point, the control unit 70 does not allow the operator to change a laser irradiation area IA beyond the margin. In other words, the control unit 70 does not allow the operator to define the area A1 within the margin area. If the operator attempts to define the laser irradiation area IA beyond the margin, the control unit 70 controls the buzzer 72 to warn the operator, and does not accept a change of the area A1.

If the switch 422 is operated, information of the circle C1 and the area A1 (information on the coordinates in the image) is stored in the memory 71. Information of the areas A1 and M1 are two-dimensional. The control unit 70 converts the area A1 into three-dimensional surgical position information on the surgical area and stores the information in the memory 71. For example, the control unit 70 rotates the area A1 relative to the central axis of the crystalline lens LE1 (here, the axis passing through the pupil center obtained from an iris IR1) as a rotation axis. Consequently, the control unit 70 interpolates a tomographic image in a direction that has not been photographed. Consequently, the control unit 70 obtains three-dimensional value information on the surgical area. Otherwise, the control unit 70 interpolates a surgical area defined on a plurality of tomographic images by an interpolation algorithm such as spline interpolation and consequently obtains three-dimensional information.

Hence, information on the surgical site that has been defined using the tomographic images is generated and stored in the memory 71. The control unit 70 controls the position detecting unit 200 to obtain reference irradiation control information to be a reference of the control information of the moving optical system on a specific disease portion included in the tomographic image. The control unit 70 associates the reference irradiation control information corresponding to a given discriminative portion with a discriminative portion in the tomographic image. The control unit 70 uses information on the irradiation pattern and the like to further generate the control information of the laser light source unit 110 when irradiating the surgical site with laser light.

In the embodiment, the control unit 70 obtains the position of the discriminative portion in the tomographic image as the reference irradiation control information by the position detecting unit 200 and associates the position with the discriminative portion in the tomographic image. Consequently, the control unit 70 associates the control information of the laser light source unit 110 with the tomographic image. The control unit 70 uses such an association relationship to generate control information of the laser irradiation unit 100 for irradiating the surgical site defined in the tomographic image with laser.

<Position Detection and Generation of Control Information>

Next, a description will be given of the acquisition of reference irradiation control information of the moving optical system on a discriminative portion of the patient's eye E, and the generation of control information of the laser light source unit 110 and the moving optical system. FIG. 5 is a diagram schematically illustrating a process of associating the surgical site with reference irradiation control information. The reference irradiation control information here is the position of the discriminative portion of the patient's eye E (the position with respect to the laser irradiation optical system 120).

The control unit 70 controls the laser light source unit 110 to set the energy of laser light to energy for position detection and emit laser light. The control unit 70 controls the scanning unit 140 (the driving parts 143 and 146) to scan the laser light in the X and Y directions (a B-scan is performed). At the same time, the control unit 70 controls the expander 130 (the driving part 135) to move (the spot of) the laser light in the Z direction. The laser light is reflected by the crystalline lens LE and enters the light receiving device 213. The control unit 70 obtains the absolute position of the anterior capsule AP of the crystalline lens LE based on the information of the driving parts 143, 146, and 135 and the intensity of a light receiving signal of the light receiving device 213.

At this point, it is preferred that the control unit 70 perform a process of estimating position detection to shorten the time of the position detection. In the control of the position detecting unit 200, the control unit 70 estimates the discriminative portion and defines the moving area of the laser spot by the moving optical system. The control unit 70 calculates a predetermined area (a three-dimensional area) corresponding to the discriminative portion (for example, the anterior capsule) included in the tomographic image. Here, the predetermined area is an area where a deviation from the discriminative portion is estimated, and is for example, an area within ±500 μm from that portion in each of the X, Y, and Z directions. The control unit 70 moves the laser spot in the calculated predetermined area and detects the position. Consequently, the time necessary for the position detecting unit 200 to detect the position can be shortened.

The control unit 70 obtains the position of the anterior capsule AP continuously or in a spaced manner (discretely). In the spaced acquisition, position information on several points is obtained. Thus, it is preferred that the control unit 70 obtain absolute position information on the curved surface of the anterior capsule AP by an interpolation process on the points, or the like (fitting). Fitting may be a polynomial expansion. In the embodiment, the control unit 70 detects the absolute position corresponding to the above-mentioned six directions of B-scans at the time of the acquisition of OCT images. The control unit 70 obtains the shape and absolute position information of the anterior capsule by fitting the detection result.

The control unit 70 controls the driving parts 143 and 146 to scan the laser light in the Y direction while controlling the driving part 135 to scan (move) the laser light in the Z direction. Consequently, the control unit 70 obtains information for position detection of the discriminative portion, the information corresponding to a B-scan in the Y direction. The control unit 70 extracts an anterior capsule AP2 to be a boundary of the tissue from the signal intensity of the light receiving signal. Consequently, the control unit 70 obtains the position of the anterior capsule AP2 (see FIG. 5), and the control unit 70 obtains the absolute position of the anterior capsule in B-scans in the other directions.

Here, for simplification of description, correction on the laser irradiation position information on the B-scan is taken as an example. In FIG. 5, the anterior capsule AP2 indicates the absolute position of the anterior capsule of the patient's eye E, the absolute position having been obtained by the position detecting unit 200. The laser light source unit 110, which emits laser light for measurement, of the position detecting unit 200 is also a light source of the laser irradiation unit 100. Hence, the position of the anterior capsule AP2 is the position of a discriminative portion for the laser irradiation optical system 120 (the laser irradiation unit 100). The control unit 70 stores in the memory 71 the control information of the moving optical system of when applying laser light to the position of the discriminative portion as reference irradiation control information relative to the discriminative portion.

The control unit 70 generates the control information of the moving optical system by association (matching) with the surgical site (which has become three-dimensional information due to the calculation) set as the reference irradiation control information. Specifically, the control unit 70 uses the discriminative portion (which has been set as the reference irradiation control information) detected by the position detecting unit 200 and the discriminative portion included in the tomographic image to associate (match) the discriminative portion of the reference irradiation control information with the discriminative portion in the tomographic image. The control unit 70 uses the irradiation pattern and the surgical condition, in addition to the reference irradiation control information, to generate control information of the moving optical system and the laser light source unit 110. The generated control information is stored in the memory 71.

In the example illustrated in FIG. 5, the control unit 70 performs the matching to obtain a distance where the anterior capsule AP1 corresponds to the anterior capsule AP2. Consequently, the control unit 70 calculates a difference on the coordinates. The control unit 70 computes a moving distance of the anterior capsule AP1 when the anterior capsule AP1 moves parallel so as to overlap with the anterior capsule AP2. The distance of parallel movement is in the Y and Z direction in FIG. 5. The control unit 70 offsets the position information of the areas A1 and M1, included in the laser irradiation position information, by the above moving distance (the movement in the Y and Z direction). The area A1 moves so as to correspond to the anterior capsule AP2 and is set as an area A2 where surgical laser light is applied. The control unit 70 moves (corrects) the circle C1 of an incision in the anterior capsule to a position on the anterior capsule AP2. The position information of the area A2 is set as an actual laser irradiation position.

Such a moving process on the diagram is the process of the control unit 70. The information on the parallel movement from the area A1 to the area A2 is generated as (part of) the control information of the moving optical system, which is based on the reference irradiation information. In terms of the control information of the moving optical system, the control unit 70 generates control information on the energy adjustment (including on/off) of the laser light source unit 110, considering the surgical condition and the irradiation pattern. Consequently, the control unit 70 generates the control information (for laser irradiation) of the moving optical system and the laser light source unit 110 (a laser light energy adjustment unit).

The above description is the correction process in the Y and Z direction. The anterior capsule AP2 is the absolute position information having a three-dimensional curved surface. The area A1 is the position information having a three-dimensional volume. The control unit 70 uses the position of the three-dimensional shape to obtain the reference irradiation control information (the anterior capsule AP2). The control unit 70 associates the information with the surgical site (the area A1) in the tomographic image and consequently generates the control information (the area A2) of the moving optical system and the laser light source unit 110.

When the matching process is complete, the control unit 70 controls the buzzer 72 to notify the operator of the completion of the process. The control unit 70 becomes enabled to accept a trigger signal by the trigger switch 410. If a trigger signal is input by the trigger switch 410, the control unit 70 controls the laser light source unit 110, the expander 130, and the scanning unit 140 based on the control information stored in the memory 71 starts laser irradiation. At the time of laser irradiation, the control unit 70 displays the state of the patient's eye E on the monitor 420 in real time. The surgical site to which laser light is applied is positioned with respect to the laser irradiation optical system 120. Consequently, a cut in the crystalline lens and an incision in the anterior capsule are made with high accuracy. The present apparatus 500 (or the control unit 70) may be configured to be capable of changing the surgical conditions such as the repetition frequency and spot size of laser upon laser irradiation.

As described above, accurate surgery can be performed by using a tomographic image and setting a surgical site. Especially, a tomographic image obtained by an apparatus, such as an OCT unit, that can photograph an intraocular state (depth information) is used. Consequently, planning (the setting of a surgical site) becomes easy. The position detecting unit 200 obtains information on a position with respect to the laser irradiation optical system, and the like. Consequently, laser irradiation with high accuracy is possible. A matching process is performed on the surgical site set based on the tomographic image with the information of the position detecting unit 200. Consequently, the position of laser irradiation is determined Consequently, the advantages of both of them are made use of. In other words, the time for the setting of a surgical site can be shortened by using a tomographic image. Furthermore, the number of positions detected by the position detecting unit is reduced and consequently the time for position detection can be shortened. Consequently, the time before surgery can be shortened. Especially, compared with a case where detailed information (for example, a tomographic image) of a patient's eye is obtained by the position detecting unit, the time can be shortened.

In the above description, the discriminative portion for position detection is the anterior capsule, but not limited thereto. It is sufficient if the discriminative portion for position detection is a portion from which information to be a reference (reference irradiation control information) can be obtained by position detection. The discriminative portion for position detection may be another discriminative portion of an eyeball, such as the posterior capsule or the corneal shape. Moreover, the discriminative portion for position detection is not limited to one curved surface such as the anterior capsule, but may include a plurality of curved surfaces. The discriminative portion for position detection may be, for example, a combination of the anterior capsule and the posterior capsule.

In the above description, the irradiation position information is obtained by parallel displacement of the surgical site information using the absolute position information. Alternatively, the irradiation position information may be obtained in a different way. The irradiation position may be obtained by performing a process such as enlargement, reduction, rotation, or distortion correction of the surgical site information.

In the above description, the position detecting unit 200 obtains the three-dimensional shape of the discriminative portion. The shape to be obtained by the position detecting unit 200 is not limited to this, but it is sufficient if the shape is a shape (information) from which the reference irradiation control information (for example, the position and center of the anterior capsule) can be obtained. The position detecting unit 200 may be configured to obtain, for example, two-dimensional information (curve).

In the above description, an OCT mage is displayed on the monitor 420 upon planning. An OCT image may not be displayed. It is sufficient if planning can be carried out with an OCT image. For example, the control unit 70 may be configured to perform image processing on the OCT image and determine a surgical site without showing the OCT image to the operator.

In the above description, the surgical site is defined as an area. However, the setting is not limited to this. It is sufficient if the position of laser irradiation is set on an OCT image and surgery is performed. For example, the operator successively specifies the positions of laser irradiation (the positions of incisions) on an OCT image displayed on the monitor 420.

In the above description, the present apparatus 500 includes the OCT unit (OCT image photographing unit) 310 as a tomographic image acquisition unit. However, the present apparatus 500 may not include the OCT unit 310. The present apparatus 500 may be configured to input image data of a tomographic image (for example, an OCT image) obtained by another apparatus. Moreover, the present apparatus 500 may include a Scheimpflug camera unit that obtains a Scheimpflug image as a tomographic image.

In the above description, the present apparatus 500 uses the laser light source unit 110 that emits surgical laser light as the laser light source of the position detecting unit 200. However, the laser light source of the position detecting unit 200 is not limited to this. It is sufficient if the laser light source of the position detecting unit 200 is a light source that can obtain a reception position of a discriminative portion of a patient's eye. The laser light source may be another unit (a second light source). In this case, it is not necessary to reduce the energy of the laser light source unit upon position detection.

In the above description, the position detecting unit 200 includes the optical system having the confocal aperture plate. However, the optical system of the position detecting unit 200 is not limited to this. It is sufficient if the optical system of the position detecting unit 200 is an optical system that can obtain absolute position information of a discriminative portion of a patient's eye. The optical system of the position detecting unit 200 may be, for example, an optical system of a confocal multiphoton excitation. In this case, the energy of the laser light source unit 110 is reduced, and two or more photons are condensed to a target position of a patient's eye. Self-fluorescence emission of a tissue by the excitation of photons occurs at the target position. The light receiving device 223 receives the light. The control unit 70 obtains the absolute position from the light receiving result similarity as described above. At this point, the confocal aperture plate is not necessarily required. Moreover, a light source that emits multiple photons is not necessarily the laser light source unit. The light receiving device 223 may be configured to receive reflected light from the target position due to the occurrence of a second harmonic wave by the multiphoton excitation. In this configuration, the laser light source unit (laser light energy adjustment unit) 110 adjusts the energy of the laser light so as to cause multiphoton absorption in eyeball tissue at the spot position of the laser light, and cause fluorescence emission or reflection at the spot position.

In the above description, the position detecting unit 200 includes the confocal optical system. However, the optical system of the position detecting unit 200 is not limited to this. It is sufficient if the optical system of the position detecting unit 200 is an optical system that can obtain the absolute position of a discriminative portion. However, the optical system of the position detecting unit 200 may be configured, for example, to obtain the absolute position of a discriminative portion of a patient's eye by time domain OCT and obtain absolute position information.

In the above description, the control unit 70 is configured to detect the position of a discriminative portion of a patient's eye using the position detecting unit 200 before surgical laser irradiation. However, the control unit 70 is not limited to this. It is sufficient if the control unit 70 is configured to obtain control information on laser irradiation by position detection of a discriminative portion. For example, the control unit 70 may be configured to detect the position of a discriminative portion using the position detecting unit 200 during surgical laser irradiation. In this case, the control unit 70 chronologically detects the position of the discriminative portion. The control unit 70 may be configured to stop laser irradiation when detecting the movement of the eyeball of a patient's eye by detecting displacement of the discriminative portion. Moreover, the control unit 70 may be configured to stop laser irradiation or change the position of laser irradiation when detecting a change in thickness of the crystalline lens during laser irradiation. The control unit 70 may be configured to appropriately obtain the control information of the moving optical system or the like.

In the above description, the present apparatus 500 is configured to perform cutting of the crystalline lens, and the like. However, the present apparatus 500 is not limited to this. It is sufficient if the present apparatus 500 is configured to perform the cutting of an eyeball tissue of a patient's eye, and the like. The present apparatus may be configured, for example, to perform cutting of the cornea, angle, iris, retina, or the like of a patient's eye.

In the above description, the ophthalmic laser surgical apparatus (the present apparatus 500) including pulse laser light is illustrated as an example of an ophthalmic laser surgical apparatus. However, the ophthalmic laser surgical apparatus according to the embodiment is not limited to this. It is sufficient if the ophthalmic laser surgical apparatus according to the embodiment is configured to perform surgery or treatment by fixing the eyeball of a patient's eye and irradiating an eyeball tissue of the fixed patient's eye with laser light. The ophthalmic laser surgical apparatus according to the embodiment may be, for example, an ophthalmic laser surgical apparatus for performing selective laser trabeculoplasty (Selective Laser Trabeculoplasity). In this case, laser light is visible pulse laser, or the like. The size of a laser spot is several hundreds micrometers. Laser is irradiated to the trabecular meshwork in the angle of the patient's eye. Laser is irradiated to the trabecular meshwork in the angle of the patient's eye. Moreover, the ophthalmic laser surgical apparatus according to the embodiment may be configured to perform photocoagulation treatment and iridotomy (iridotomy). In this case, the laser light source may be a pulse laser light source or a continuous wave laser light source. In a case of performing photocoagulation treatment on the retina, the OCT unit may be configured to photograph a tomographic image of the posterior segment.

The ophthalmic laser surgical apparatus according to the present disclosure is not limited to the above embodiment, but various modifications can be made thereto. The ophthalmic laser surgical apparatus according to the present disclosure includes such modifications within a range where the technical idea is identical.

The ophthalmic laser surgical apparatus according to the embodiment can be represented as the following apparatuses.

In other words, the first ophthalmic laser surgical apparatus includes a laser light source configured to emit surgical laser light, a laser irradiation optical system being a laser irradiation optical system configured to irradiate a target position with laser light emitted from the laser light source and having a moving optical system configured to move a spot of the laser light three-dimensionally, a tomographic image acquisition unit configured to obtain a tomographic image of a patient's eye, a surgical site determining unit configured to set a surgical site based on the tomographic image obtained by the tomographic image acquisition unit, an irradiation position information acquisition unit configured to obtain irradiation position information of the laser irradiation optical system for at least one discriminative portion included in the tomographic image, a control information generating unit configured to obtain an association relationship between the irradiation position information obtained by the irradiation position information acquisition unit and the tomographic image and generate control information of the laser irradiation optical system for applying laser to the surgical site set by the surgical site determining unit, using the obtained association relationship, and an irradiation control unit configured to control the moving optical system based on control information generated by the control information generating unit, and irradiate a predetermined surgical site with laser light to perform surgery.

The second ophthalmic laser surgical apparatus according to the first ophthalmic laser surgical apparatus has the irradiation position information acquisition unit configured to share the laser irradiation optical system, include a second light source to emit light for acquisition of irradiation position information, and a light receiving device to receive light from a spot position where the light emitted from the second light source is condensed, and the irradiation position information acquisition unit configured to move a spot of the light emitted from the second light source using the moving optical system, and obtain the irradiation position information based on a light receiving signal from the light receiving device.

The third ophthalmic laser surgical apparatus according to an ophthalmic laser surgical apparatus for irradiating a patient's eye with laser light and performing surgery, and includes a laser light source configured to emit surgical laser light, a laser irradiation optical system being a laser irradiation optical system configured to irradiate a target position with the laser light emitted from the laser light source and having a condensing optical subsystem configured to condense the laser light, and a moving optical system configured to move a condensed spot position three-dimensionally, a tomographic image acquisition unit configured to obtain a tomographic image, a reference irradiation control information acquisition unit configured to obtain as reference irradiation control information control information of the laser irradiation optical system, the control information corresponding to at least one discriminative portion included in the tomographic image, a surgical site determining unit configured to set a surgical site based on the tomographic image obtained by the tomographic image acquisition unit, an irradiation information control unit configured to generate control information of the laser irradiation optical system for performing laser irradiation on the surgical site set by the surgical site determining unit based on the reference irradiation control information obtained by the reference irradiation control information acquisition unit, and an irradiation control unit configured to control the moving optical system based on the control information generated by the irradiation control information generating unit, and irradiate a predetermined surgical site to perform surgery.

The fourth ophthalmic laser surgical apparatus according to the third ophthalmic laser surgical apparatus has the reference irradiation control information acquisition unit configured to share the laser irradiation optical system and include a second light source for acquisition of the reference irradiation control information, and a light receiving device to receive light from a spot position where light emitted from the second light source is condensed, and obtain the reference irradiation control information based on a light receiving signal from the light receiving device, the light receiving signal being obtained while the spot position is moved using the moving optical system

The fifth ophthalmic laser surgical apparatus according to the fourth ophthalmic laser surgical apparatus has the laser light source being a pulse laser light source configured to emit pulse laser light to cause a breakdown at the spot position of the laser light, be shared as the second light source, and include a laser light energy adjustment unit configured to adjust the energy of the laser light so as not to substantially cause a breakdown at the spot position where the laser light emitted from the laser light source is condensed.

The sixth ophthalmic laser surgical apparatus according to the third ophthalmic laser surgical apparatus has the reference irradiation information acquisition unit configured to calculate a predetermined area corresponding to the discriminative portion included in the tomographic image, and obtain the reference irradiation control information on the calculated predetermined area.

The seventh ophthalmic laser surgical apparatus according to the fourth ophthalmic laser surgical apparatus has the surgical site determining unit including a monitor configured to display a tomographic image of the patient's eye, and an input unit configured to input a signal for setting the surgical site, or a check unit configured to check the surgical site, on the tomographic image of the patient's eye displayed on the monitor.

The eighth ophthalmic laser surgical apparatus according to the fourth ophthalmic laser surgical apparatus has the reference irradiation control information acquisition unit including a confocal optical system where a light receiving part of the light receiving device is conjugated with the spot position of the laser light.

The ninth ophthalmic laser surgical apparatus according to the eighth ophthalmic laser surgical apparatus has the reference irradiation control information acquisition unit configured to match a confocal image obtained by the confocal optical system and the tomographic image obtained by the tomographic image acquisition unit, and associate the control information of the moving optical system and a coordinate position in the tomographic image.

The tenth ophthalmic laser surgical apparatus according to the fifth ophthalmic laser surgical apparatus includes a laser light energy adjustment unit configured to adjust energy so as to cause multiphoton absorption in an eyeball tissue at the spot position of the laser light and cause fluorescent emission or reflection at the spot position, and has the light receiving device configured to receive fluorescent or reflected light caused at the spot position.

The eleventh ophthalmic laser surgical apparatus according to the third ophthalmic laser surgical apparatus has the tomographic image acquisition unit configured to obtain at least two tomographic images whose positions are different, and the reference irradiation control information acquisition unit configured to obtain irradiation position information in directions corresponding to the at least two tomographic images.

According to the first to eleventh ophthalmic laser surgical apparatuses, it is possible to set a surgical site using a tomographic image and perform surgery with high accuracy.

The foregoing detailed description has been presented for the purposes of illustration and description. Many modifications and variations are possible in light of the above teaching. It is not intended to be exhaustive or to limit the subject matter described herein to the precise form disclosed. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims appended hereto.

What is claimed is:

1. An ophthalmic laser surgical apparatus comprising:
a laser light source configured to emit surgical laser light;
a laser irradiation optical system including a condensing optical system configured to form a spot of the laser light on a patient's eye by condensing the laser light, and a moving optical system configured to move a position of the spot three-dimensionally;
a tomographic image acquisition unit configured to obtain a tomographic image of the patient's eye;
a reference irradiation control information acquisition unit configured to obtain, as reference irradiation control information, control information of the laser irradiation optical system, the control information corresponding to at least one discriminative portion included in the tomographic image;
a surgical site determining unit configured to determine a surgical site based on the tomographic image obtained by the tomographic image acquisition unit;
an irradiation control information generating unit configured to generate control information of the laser irradiation optical system for applying the laser to the surgical site determined by the surgical site determining unit based on the reference irradiation control information obtained by the reference irradiation control information acquisition unit; and
an irradiation control unit configured to control the laser irradiation optical system based on the control information generated by the irradiation control information generating unit to allow the laser irradiation optical system to apply the laser light to a predetermined surgical site of the patient's eye.

2. The ophthalmic laser surgical apparatus according to claim 1, wherein
the reference irradiation control information acquisition unit includes:
at least one component of the laser irradiation optical system;
a second light source configured to obtain the reference irradiation control information; and
a light receiving device configured to receive reflected light from a spot position of light emitted from the second light source, and
the reference irradiation control information acquisition unit obtains the reference irradiation control information based on a light receiving signal from the light receiving device, the light receiving signal being obtained while the spot position is moved using the moving optical system.

3. The ophthalmic laser surgical apparatus according to claim 2, further comprising a laser light energy adjustment unit configured to adjust energy of the laser light emitted from the laser light source, wherein
the laser light source is a pulse laser light source configured to have an ability to emit pulse laser light to cause a breakdown at the spot position of the laser light, and also serve as the second light source, and
the laser light energy adjustment unit adjusts the energy of the laser light emitted from the laser light source to substantially free of a breakdown at the spot position of the laser light when the laser light source is used as the second light source.

4. The ophthalmic laser surgical apparatus according to claim 1, wherein the reference irradiation information acquisition unit calculates a predetermined area corresponding to the discriminative portion included in the tomographic image, and obtains the reference irradiation control information on the calculated predetermined area.

5. The ophthalmic laser surgical apparatus according to claim 2, wherein the surgical site determining unit includes:
a monitor configured to display a tomographic image of the patient's eye; and
an input unit configured to determine the surgical site using the tomographic image of the patient's eye displayed on the monitor.

6. The ophthalmic laser surgical apparatus according to claim 2, wherein a position of a light receiving part of the light receiving device of the reference irradiation control information acquisition unit and the spot position of the laser light from the second light source of the reference irradiation control information acquisition unit are in a substantially conjugate relationship.

7. The ophthalmic laser surgical apparatus according to claim 6, wherein the irradiation control information generating unit matches the reference irradiation control information obtained by the reference irradiation control information acquisition unit with the discriminative portion included in the tomographic image obtained by the tomographic image acquisition unit, and
the irradiation control information generating unit associates control information of the moving optical system with a coordinate position in the tomographic image based on the matching result.

8. The ophthalmic laser surgical apparatus according to claim 3, further comprising a laser light energy adjustment unit configured to adjust the energy of the laser light source, wherein
the laser light energy adjustment unit is configured to adjust the energy of the laser light to a level that causes an eyeball tissue of the patient's eye to absorb a multiphoton at the spot position of the laser light, and to cause fluorescent emission or reflection at the spot position, and
the light receiving device receives fluorescent or reflected light caused at the spot position.

9. The ophthalmic laser surgical apparatus according to claim 1, wherein
the tomographic image acquisition unit obtains a plurality of tomographic images corresponding to different positions of the patient's eye, and
the reference irradiation control information acquisition unit obtains the reference irradiation control information based on the plurality of tomographic images.
